(19) United States
(12) Patent Application Publication
(10) Pub. No.: US 2014/0081247 A1
(43) Pub. Date: Mar. 20, 2014

(54) PROJECTOR DEVICE WITH SELF-CORRECTING FUNCTION, AND MEDICAL DEVICE COMPRISING THE PROJECTOR DEVICE

(71) Applicants: Kurt Heiberger, Nuernberg (DE); Andreas Schnalke, Nuernberg (DE)

(72) Inventors: Kurt Heiberger, Nuernberg (DE); Andreas Schnalke, Nuernberg (DE)

(73) Assignee: EYESIGHT & VISION GMBH, Nuernberg (DE)

(21) Appl. No.: 14/003,112

(22) PCT Filed: Feb. 28, 2012
(86) PCT No.: PCT/EP2012/053308

§ 371 Date: Nov. 25, 2013

(30) Foreign Application Priority Data
Mar. 4, 2011 (DE) 10 2011 001 083.1

(51) Int. Cl. A61B 3/10; A61F 9/008

(52) U.S. Cl. CPC A61B 3/1015; A61F 9/008
USPC 606/4; 351/211

(57) ABSTRACT

The aim of the present invention is to provide a projector device and a medical device with said projector device, said projector device facilitating an improved measurement of the local refractive power of an optical body, in particular an eye.

For this purpose, the invention proposes a projector device for projecting a two-dimensional pattern on a plane, in particular in a human eye, with at least one light source (LS1, LS2) which generates a light beam, with a deflecting device (MSS) which facilitates a deflection of the light beam at a deflection angle in order to generate the two-dimensional pattern on the plane, with a beam path which extends at least from the light source (LS1, LS2) to the plane, with at least one sensor device (L1, D1, W2, D2) which is designed to determine the actual state of a wavefront of the light beam at any position of the beam path, and with at least one correcting device (EL; O2, O3) which is arranged in the beam path and which facilitates an alteration of the wavefront of the light beam. In accordance with the invention, a position-resolved alteration of the wavefront is carried out by an optical control element with a variable focal length.

The invention relates to a projector device for projecting a two-dimensional pattern on a plane, in particular in a human eye, with at least one light source which generates a light beam, with a deflecting device which facilitates a deflection of the light beam at a deflection angle in order to generate the two-dimensional pattern on the plane, with a beam path which extends at least from the light source to the plane, with a sensor device which is designed and arranged to determine the actual state of a wavefront of the light beam at any position of the beam path, with an evaluation unit which is designed to record the actual state of the wavefront, and with a correcting device which is arranged in the beam path, and which facilitates an alteration of the wavefront of the light beam. The invention also relates to a medical device with the projector device.

Today it is possible to correct the defective vision of patients through laser-supported methods of treating the cornea, wherein the surface shape of the cornea is changed through laser ablation of the surface of the cornea so that the ametropia is corrected. Such treatment methods comprise first a measurement of the patient's ametropia and second the correction of the cornea using a treatment laser.

For example, the publication DE102006005473A1, which is probably the closest prior art, discloses an apparatus for measuring optical aberrations in the human eye, said apparatus being, in one possible embodiment, integrated in a treatment laser in order to check the result of a treatment in situ in cases of individual adjustment of contact lenses or intraocular lenses or of surgical correction of the cornea and to calculate a necessary correction if required. The apparatus comprises a laser diode for determining the local refractive power in the eye, wherein a surface scan is performed by guiding the light beam of the laser diode across the eye via a tiltable mirror which is implemented as a scanning micromirror. The eye test character to be displayed is adjusted to the mean refractive power of the eye by positioning the scanning micromirror on an electrically controlled slide so that the measuring beam of the laser diode can be precompensated in the region of under 0.1 dpt. Before the refractive power of the eye is measured, the eye is fixed so that it is measured in a relaxed state. For this purpose, a flashing dot or a ring is projected as an eye test character into the eye and the eye adapts to it. The eye test character is generated using a light beam which is deflected by the scanning micromirror as well.

By controlling the light, source and the tiltable mirror under consideration of the previously measured local refractive power (coma, astigmatism) of the eye, the eye test character can be projected so that it is displayed undistorted in the eye. To ensure a consistently high measurement accuracy over the entire measurement range, the divergence of the laser beam supplied by the laser diode is adjusted with an electrically controlled liquid lens as a function of the ametropia of the eye to be measured, thereby ensuring a constant beam diameter of less than 200 μm.

The aim of the present invention is to provide a projector device and a medical device with said projector device, said projector device facilitating an improved measurement of the local refractive power of an optical body, in particular an eye.

This problem is solved by a projector device with the features of claim 1 and by a medical device with the features of claim 15. Preferred or advantageous embodiments of the invention are derived from the subclaims, the following description and the accompanying figures.

Within the scope or the invention, a projector device is proposed which is suitable and/or designed to project a two-dimensional pattern in a plane. The plane is preferably arranged in or on a human eye. Specifically, the projector device is designed such that the two-dimensional pattern is projected on the retina of the eye as a plane. Specifically, the projector device is designed such that the two-dimensional pattern is projected on the macula of the retina. The two-dimensional pattern may be designed, tor example, as a symbol, a circle, a character, a numeral, an image or a structure.

In some embodiments, the projector device ensures that two-dimensional patterns are projected on the plane undistorted and/or in sharp focus. In particular, the image must be without optical aberrations. In other embodiments, the projector device is designed just to improve the optical quality in a partial region of the beam path. The projector device comprises at least one light source which generates a light beam. In alternative embodiments, several light sources may be present. If the two-dimensional pattern is intended to be visible to the patient, the light beam preferably has a wavelength in the visible range, for example between 400 μm and 800 μm. If the two-dimensional pattern is intended to be invisible, a light beam in the invisible near-infrared range, for example between 780 nm and 1400 nm, may be utilized. Combinations and superimpositions of several light beams or light sources are possible.

The projector device comprises a deflecting device which implements a deflection of the light beam at a deflection angle in order to generate the two-dimensional pattern on the plane. Particularly preferably, the deflecting device facilitates a deflection into two independent directions. It is particularly preferable that the deflecting device is designed as a scanner device with a scanning mirror, but it can also be implemented using a moving prism or a moving lens in other embodiments.

A beam path, which may be bent or angled if required, extends between the light source and the plane, wherein said beam path may also extend further, in particular beyond the plane. In particular, the beam path may include regions which are arranged behind the plane in an optical forward direction from the light source to the plane, said regions comprising reflections or scatterings from the plane.

At least one sensor device is arranged at a position within the beam path, said position being an arbitrary position in the most generic embodiment, with said sensor device being designed to determine the actual state of a wavefront of the light beam.

Since the light beam is deflected via the deflecting device in order to generate the two-dimensional pattern on the plane, the sensor device can record only the wavefront of the light beam currently hitting the sensor device or the backscattering of said light beam. If the wavefronts of all chronologically successive light beams forming the two-dimensional pattern are registered, said wavefronts can be used to determine an extended wavefront.

Different actual states of the wavefronts of the light beam can result, in particular, from different divergences of the light beam or from different directions of propagation, in particular angles of the light beam. In particular, the sensor device is designed as an aberrometer, a Shack-Hartmann sensor, a Tschernig aberrometer or a wavefront analyzer or as a simplified embodiment of said devices. The actual state of the wavefront may be registered by the sensor device using an absolute measurement reading. However, it is also possible to register the actual state using a relative value. For example, the actual state may be regarded as a position of the light beam's point of impact on a plane, wherein the position forms a relative measurand of the actual state of the wavefront but does not allow an absolute measurement reading to be indicated for the actual state of the wavefront.

A correcting device is integrated into the beam path, said correcting device facilitating an alteration of the wavefront of the light beam. In particular, the correcting device is arranged in front of the sensor device in the light beam's direction of propagation, so that alterations of the wavefront of the light beam can be registered by the at least one sensor device.

As a further component, the projector device comprises an evaluation unit which can record the actual state of the wavefront and compare the actual state to a target state of the wavefront of the light beam. Referring to the previous example, a target state of the light beam may be regarded as a further position of the light beam on a sensor surface or as another relative or absolute value.

In accordance with the invention, it is proposed that the correcting device comprise an optical control element with controllable, preferably electrically controlled, focal length, and that the evaluation unit control the optical control element so that the actual state tracks the target state in a position-resolved way.

The evaluation unit implements a supervisory circuit, in particular a regulator circuit or control circuit, which transfers the measured actual state of the wavefront into the target state in a position-resolved way using the optical control element in order to perform a self-correction of the projector device. Particularly preferably, the position-resolved control is understood as being location-dependent with respect to the plane and/or angle-dependent with respect to the deflection angle.

The particular advantage of the invention is that a self-correction of the beam path or at least of a section of the beam path can be achieved by way of the evaluation unit and the tracking through the optical control element. This makes it possible to generate on the plane a two-dimensional pattern that has no optical aberrations or at least only few optical aberrations, or, if the at least one sensor device is arranged in front of the plane in the beam path, to compensate for or correct optical aberrations created by optical elements of the projector device in the path to the measuring point. For example, it is possible to use cheap optical components in the projector device or to use components that permit higher tolerances in positioning, since imaging or adjustment errors that may occur can be compensated for through tracking or self-correction.

This can be achieved, in particular, by using an optical adjusting element with electrically controllable focal length that not only compensates for a distortion of the two-dimensional pattern on the plane but also adjusts for a divergence or an angular error of the light beam, so that even spherical optical aberrations and/or a defocus, meaning a lack of focus on the plane, in particular on the retina, can be compensated for.

In a possible embodiment, the recording or the actual state can be performed once as a reference measurement or initialization measurement, for example at the time when the projector device is first initialized or mounted or when a calibration becomes necessary. During further operation, the tracking or self-correction is carried out on the basis of the actual states originally measured, for example using a control circuit. This can be sufficient because optical aberrations caused by maladjustment or cheap optical components are static and need not be constantly monitored.

Particularly preferably, however, the self-correction, meaning the recording of the actual states and the tracking, is performed constantly in order to detect and correct changes in the beam path immediately, for example using a regulator circuit. Particularly preferably, the actual state of the wavefront is recorded at least once per second, preferably at least five times per second, in particular at least ten times per second or continuously, so that the self-correction is performed in real time.

In a particularly preferred embodiment of the invention, the two-dimensional pattern is designed as an accommodation target, in particular an eye test character, for the eye. Projecting an accommodation target onto the eye, in particular onto the retina, facilitates a stabilization of the viewing direction, since the patient can be instructed to look in the (perceived) direction where the object shown to the patient via the accommodation target originates. This allows for measurements within the visual axis. The self-correction of partial sections of the beam path or of the entire beam path leads to improved image sharpness of the accommodation target or the retina, so that the eye can comfortably adjust to wide distances or to infinity, thereby entering a mostly relaxed condition. In this state, the eye can be measured with particular accuracy and also surgically treated with particular exactness.

In a particularly preferred constructional embodiment of the invention, the optical control element is designed as a an adaptive, in particular slidable and/or deformable lens. By applying an electrical control signal, for example a control voltage, to the lens and/or by controlling the lens in any corresponding way, the focal length of said lens can be set. Preferably, the lens has a free diameter of more than 2 mm, more preferably of more than 5 mm, wherein the large free diameter is advantageous because light beams which pass the lens off the center of the lens will still perceive the lens as an approximately ideal lens and transmit through it with few optical aberrations or without optical aberrations.

Alternatively, the optical control element may be designed as an adaptive mirror wherein the focal length is adjusted by changing the curvature of the mirror surface. This embodiment is mechanically easier to implement but requires more installation space.

In a preferred embodiment of the invention, the deflecting device is designed as a scanning micromirror, said scanning micromirror particularly preferably facilitating a deflection at a deflection angle into two perpendicular directions. In particular, it will be an X-Y scanning mirror. Employing oscillation frequencies in the range from 100 Hz to 110 kHz, the scanning micromirror allows for a comprehensive scan of the plane used for projecting the two-dimensional pattern. In particular, the scanning micromirror can be deflected in two dimensions. The scanning micromirror has a free mirror surface with a diameter of less than 7 mm, preferably less than 3 mm. Owing to its small size, the scanning micromirror can be operated in a resonance mode with the frequencies mentioned, so that the scanning micromirror always performs the same deflections in the same order irrespective of the shape of the two-dimensional pattern, said two-dimensional pattern being generated by activation or deactivation of the light source or the light beam. The resonance mode makes it very easy to control the scanning micromirror since the manner of controlling it does not need to be adapted to the two-dimensional pattern.

From a constructional point of view, the use of the adaptive lens and the scanning micromirror makes a design with very compact dimensions possible, so that the projector device can be easily integrated into any medical instrument.

Preferably, the optical control element can be controlled using the same frequencies as used for the deflecting device or be operated synchronously with the deflecting device, since a different focal length of the optical control element needs to be set for each angular position of the deflecting device.

It is particularly preferred that the way in which the actual state tracks the target state is achieved exclusively by controlling the optical control element. In principle it is also possible, at least with respect to the higher-level optical aberrations, to achieve the difference between the actual state and the target state by controlling the deflecting device or by activating or deactivating the light source, as is described in the publication DE102006005473A1 referred to at the beginning. Particularly preferably, the tracking is achieved in the present invention by controlling the optical control element exclusively, meaning that if deviations between actual state and target state are measured, tracking will not be achieved by controlling the light scarce or the deflecting device. The reason for this is the ability of the correcting device to compensate the wavefront in a position-resolved way, so that higher-level optical aberrations such as coma or astigmatism etc. can be compensated for, just as corrections of spherical optical aberrations or defocus can be performed. Control and/or regulation of the projector device are simplified by the fact that only one actuator, namely the optical control element, needs to be addressed.

In a particularly preferable embodiment, the light source is designed as at least one laser beam source. In particular, laser diodes are utilized. The advantage of a laser beam source is that the beam quality is very high and the beam can be guided through the projector device with a small diameter. For example, the beam diameter (FWHM) is smaller than 500 μm, in particular smaller than 300 μm. This makes it possible to use optical components, such as the scanning micromirror or the optical control element, that are very small with regard to their optically active diameter (lens surface, mirror surface). This in turn allows the entire projector device to be constructed as a very small unit which, as will be explained below, can be inserted as a compact component into a medical instrument.

In a preferred embodiment of the invention, the light source or laser beam source is designed to emit colored and/or polarized light. By using colored light sources or several light sources with different colors, it becomes possible to display a multicolored two-dimensional pattern which creates a pleasant sensation for the patient. The use of polarized light, in particular horizontally or vertically polarized light, makes it possible to reflect or transmit the light beam selectively within the beam path through a combination of mirror polarizers and lambda plates, thus shaping the beam path in a flexible way.

In a particularly preferred embodiment of the invention, the at least one sensor device is arranged in the beam path so that it receives a backscattering of the light beam from the plane. If we again regard the beam path in the direction of the light beam, the sensor device is arranged behind the plane. This embodiment is advantageous because any optical aberrations will accumulate or sum up in the beam path on the way from the light source to the plane and the sensor device with thus record an actual state which incorporates any optical aberrations of the beam path. Consequently, all optical aberrations will be compensated for by having the actual state track the target state.

If the plane is located within the human eye, in particular on the retina, then the eye, which in this case would usually be the optical element with the largest amount of optical aberrations, will also be measured as a whole or in parts by the sensor device. As the actual state tracks the target state, it can be ensured that the two-dimensional pattern is displayed on the plane with few optical aberrations or without optical aberrations.

In a possible alternative or further form of the invention, the at least one sensor device or a further sensor device is arranged in front of the plane if we regard the beam path in the direction of the light beam, making it possible to receive or to measure a light beam incident towards the plane. In this configuration, the sensor device records the actual state of a partial region of the beam path, with the plane and specifically the human eye being intentionally omitted as an optical element in the beam path. In this embodiment of the invention, optical aberrations that occur, for example, independently of the human eye in the beam path can be compensated for when the actual state tracks the target state. In this case it is possible, for example, to insert inexpensive optical components, such as components made of plastic, in front of the plane or to utilize optical components in the peripheral regions of the aperture, with the evaluation unit having the ability to compensate for any optical aberrations that may occur. A light beam with few optical aberrations or without optical aberrations will be used for projecting the two-dimensional pattern on the plane, so that any optical aberrations that then occur will be caused by the eye.

In a particularly preferred embodiment of the invention, one sensor device is arranged in front of the plane and another sensor device is arranged behind the plane, with the sensor device in front of the plane being designed to register the actual state of a partial region of the beam path and the sensor behind the plane being designed to register the actual state behind the plane. The advantage of such double registering is that an estimation can be performed as to which optical aberrations are present in front of the plane and which optical aberrations behind the plane. As a result, the optical aberrations measured in front of the plane can be assigned to the projector device. By subtracting the optical aberrations in front of the plane from the optical aberrations behind the plane, a good approximation of the optical aberrations which can be assigned to the eye is achieved, thereby defining a distribution of the local refractive power in the eye. It is thus possible to implement a measurement of the optical properties of the human eye while using the projector device and determining the actual state or having the actual state track the target state.

In a constructional implementation of the invention, the sensor device or the further sensor device is arranged in the beam path after a collimating lens. The collimating lens is one of the important components in the beam path, so that it seems sensible to check the beam quality after the collimating lens.

In a possible implementation of the invention, the sensor device is designed as a wavefront measuring instrument. Wavefront measuring instruments are generally known in the art, for example in the form of the Shack-Hartmann sensor or the Tschernig aberrometer known to persons skilled in the art.

In another embodiment of the invention, the sensor device comprises a position sensitive diode (PSD), a quadrant detector as a sensor, or is designed as another kind of flat sensor, such as a CCD chip or a CMOS chip, wherein the sensor device is preferably arranged in an image plane of the beam path. In principle, the two-dimensional pattern should be displayed on the sensor in the image plane, wherein light beams mapped onto regions which lie outside the ideal image of the two-dimensional pattern or outside one's own ideal image pixel indicate a deviation of the actual state from the target state of the wavefront of the light beam, which can be compensated for by controlling the optical control element. The size of the deviation or the distance of the point of impact of the light beam on the sensor from the ideal image or image pixel is a relative value or a relative measurand of the actual state of the wavefront.

Particularly preferably, the projector device is designed as a binocular device in order to simultaneously project the two-dimensional pattern into both eyes of a patient. This embodiment may be designed such that one sensor device is arranged behind each plane in order to facilitate measuring both eyes independently of each other.

To further improve the patient comfort during examination or treatment, the two-dimensional pattern may form a 3D image using the binocular device. This can be achieved by projecting two different two-dimensional patterns into the eyes of the patient, said patterns being designed such that they are experienced as a 3D image when viewed at the same time.

A particularly preferred position of the correcting device would be directly in front of or directly behind the collimating lens or one of the collimating lenses or in an eyepiece of the projector device. It is also possible to have one correcting device near the collimating lens and a second correcting device in the eyepiece or even correcting devices in each of the eyepieces. In the latter case, the partial region of the beam path up to a measurement position of the first of the sensor devices can be corrected via a first regulatory circuit by controlling the correcting device near the collimating lens, and the two-dimensional pattern in the eye or in the eyes can be corrected by controlling the correcting device in the eyepiece or in the eyepieces.

In a preferred embodiment of the invention, the control device is arranged in a region between the deflecting device and a collimating lens. The collimating lens is designed, in particular, such that the light beam coming from the deflecting device is shaped parallel to an optical axis of the beam path. Preferably, the distance between the collimating lens and the deflecting device corresponds to the focal length of the collimating lens. Arranging the control device in the abovementioned region is advantageous because the deflection angle of the light beam after the control device can be actively changed by controlling the control device. This makes it possible to change the position of the light beam on the subsequent optical components and/or on the plane, in particular in the eye, actively.

In a possible further form of the invention, an invisible laser beam is emitted as a light beam from the supplementary light source or from one of the supplementary light sources. The invisible laser beam can be arranged in the wavelength range of UV light or NIR light, for example. Using an invisible laser beam is advantageous because the entire surface of the plane can be scanned by the invisible laser beam regardless of the visible pattern actually projected on the plane, thus making it possible to record the actual states on the entire surface. This solves a problem that may arise, for example, if the two-dimensional pattern has the shape of a ring, which requires the visible light beam to be switched off inside the ring and outside the ring, consequently making it impossible to register the actual state there. In such a case, the entire scanned surface of the plane can be measured by evaluating the invisible laser beam, which may be permanently activated if required.

A further object of the invention relates to a medical device with a projector device of any one of the preceding claims. The medical device may be designed as a topography measuring instrument, a wavefront measuring instrument or a layer thickness measuring instrument or even intraoperatively in refractive eye surgery, for example as a treatment laser for surgically correcting the refractive power of the eye.

In a preferred further embodiment of the medical device in the form of a treatment laser, the treatment laser is guided parallel to or along the same axis as the light beam so that the changes made by the treatment laser can be recorded in situ. The advantage of this embodiment is, in particular, that not only the light beam is guided into the eye with few optical aberrations or without optical aberrations but that the treatment laser beam, since it is guided along the same axis into the eye, also has only few optical aberrations or no optical aberrations

Other features, advantages and effects of the present invention are derived from the following description of preferred embodiments of the invention and the accompanying figures. The figures show:

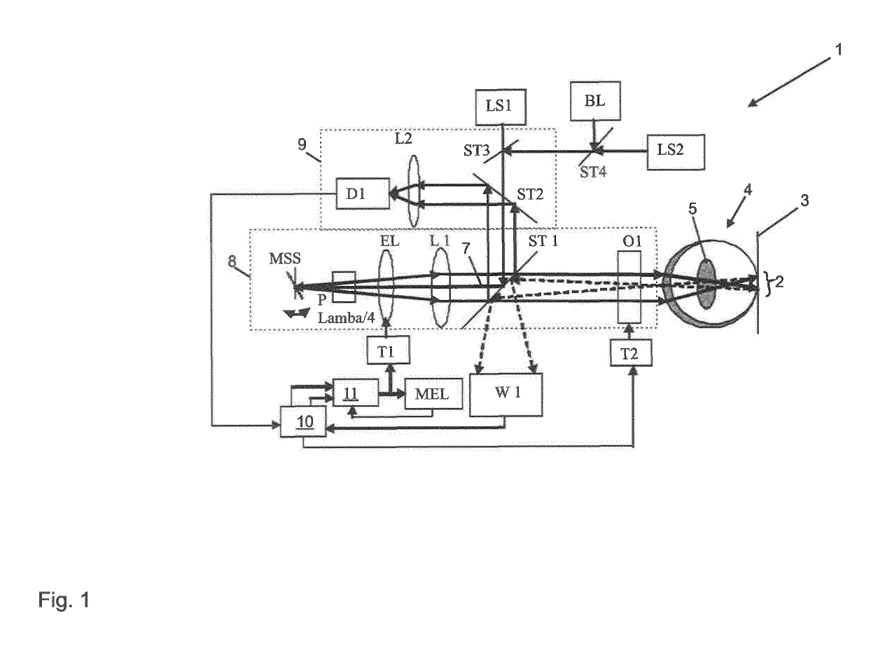

FIG. 1 a schematic block diagram of a first embodiment of the invention;

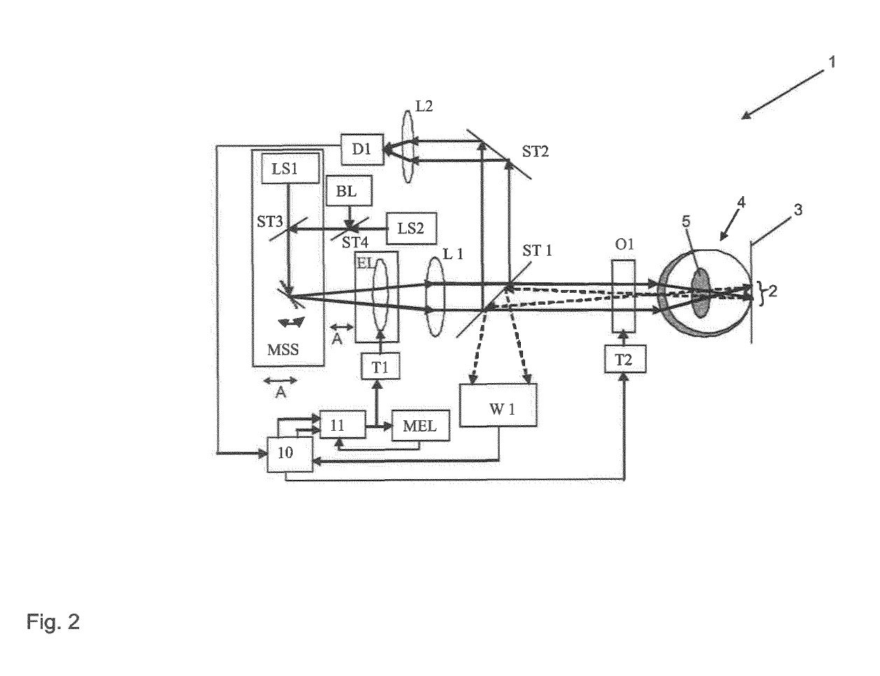

FIG. 2 a schematic block diagram of a second embodiment of the invention;

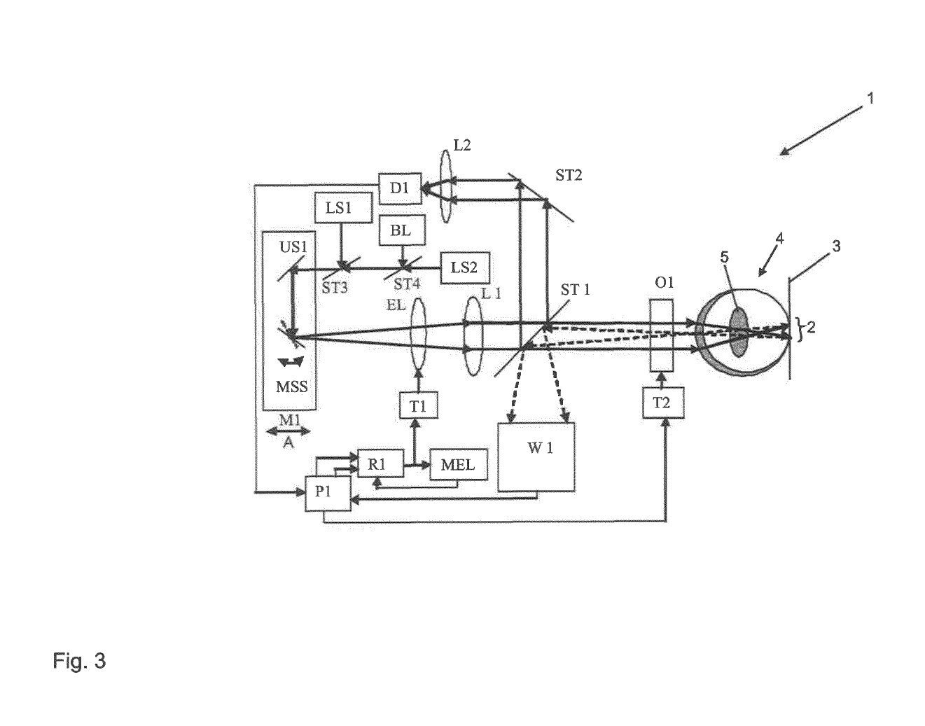

FIG. 3 a schematic block diagram of a third embodiment of the invention;

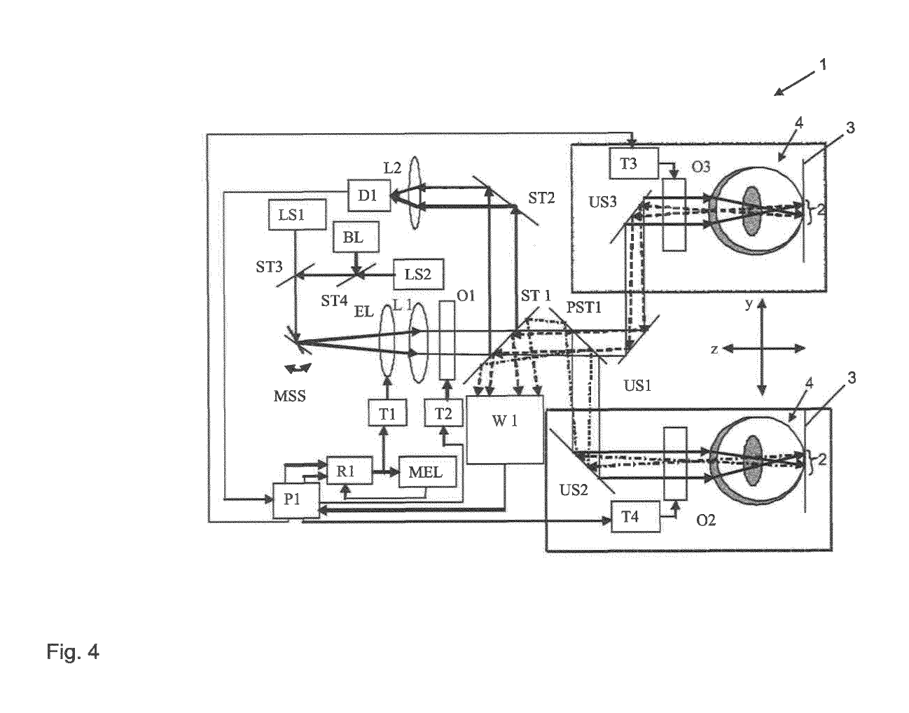

FIG. 4 a schematic block diagram of a fourth embodiment of the invention;

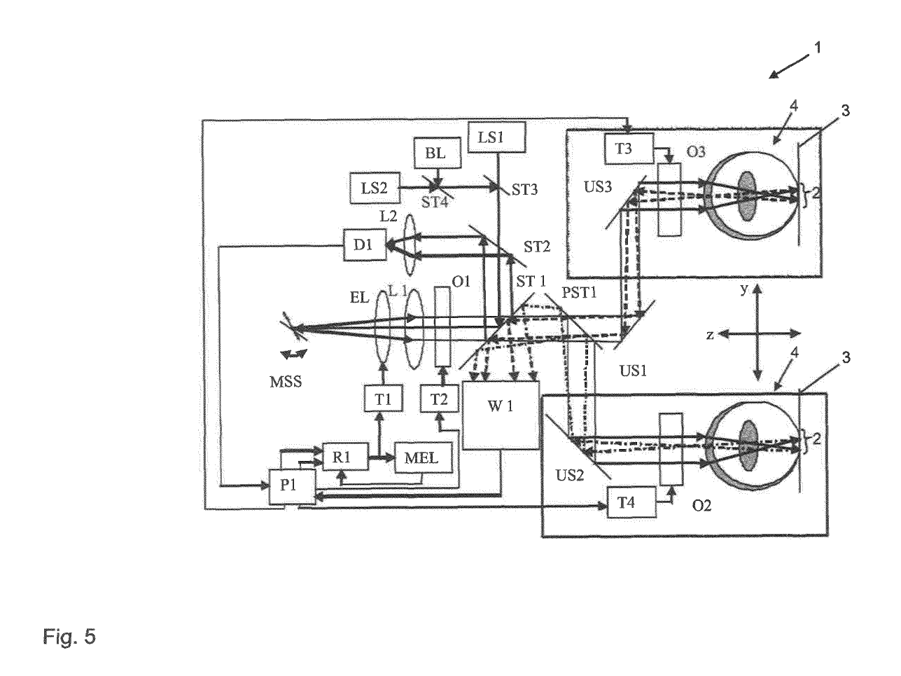

FIG. 5 a schematic block diagram of a fifth embodiment of the invention;

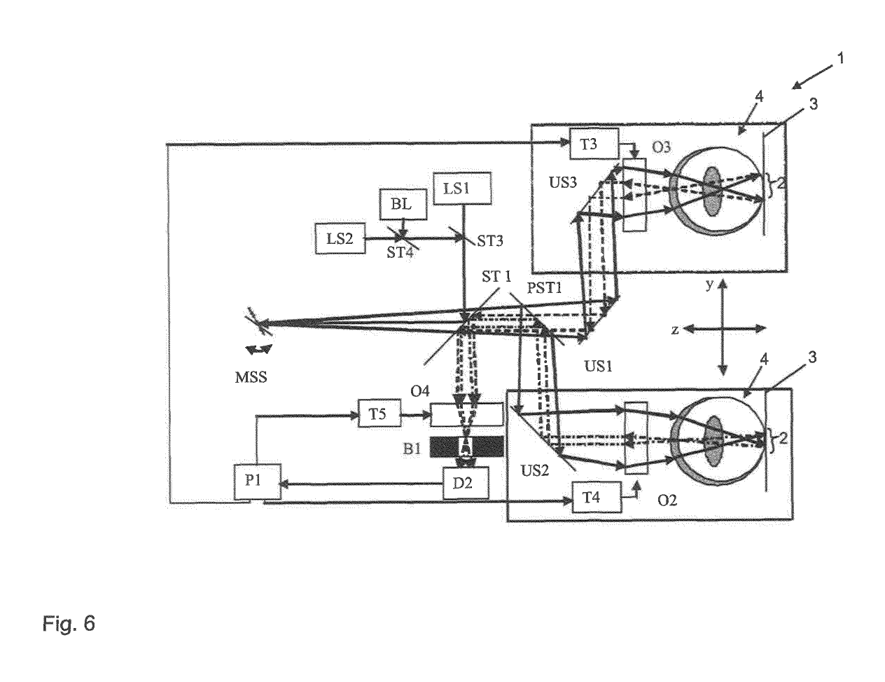

FIG. 6 a schematic block diagram of a sixth embodiment of the invention;

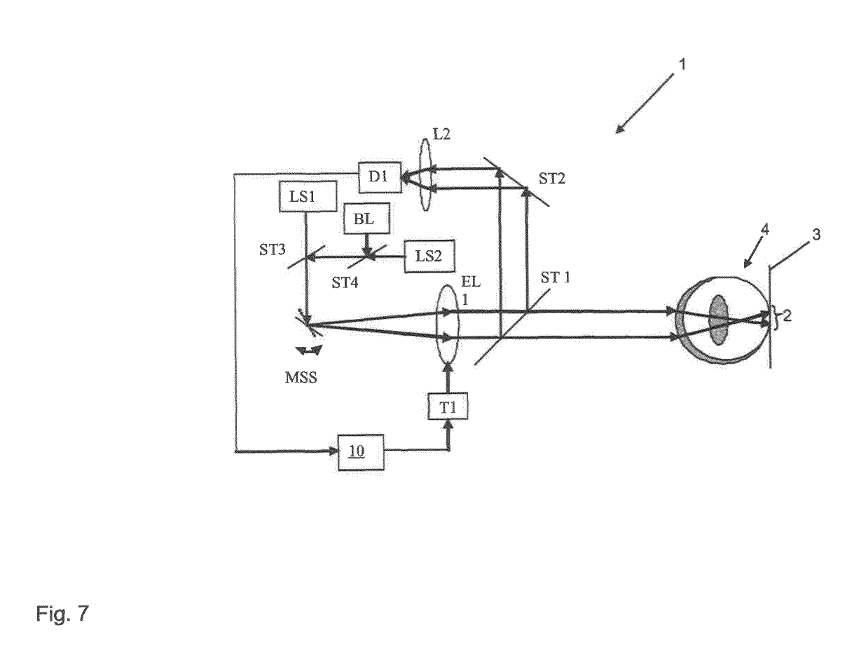

FIG. 7 a schematic block diagram of a seventh embodiment of the invention;

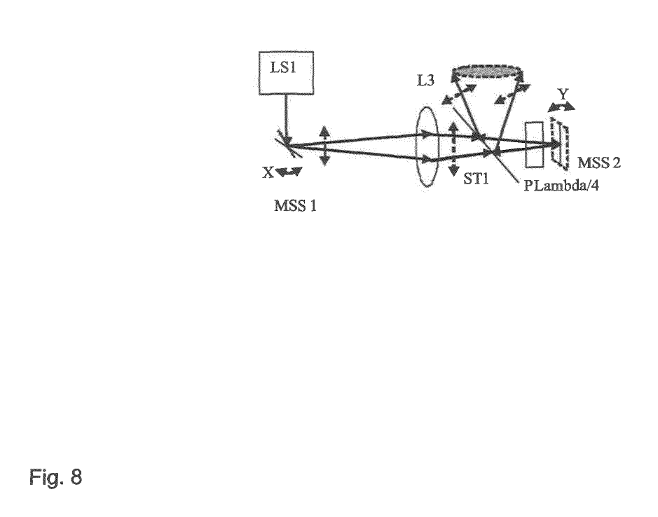

FIG. 8 a schematic block diagram of a first modification of the scanning micromirror of the preceding figures;

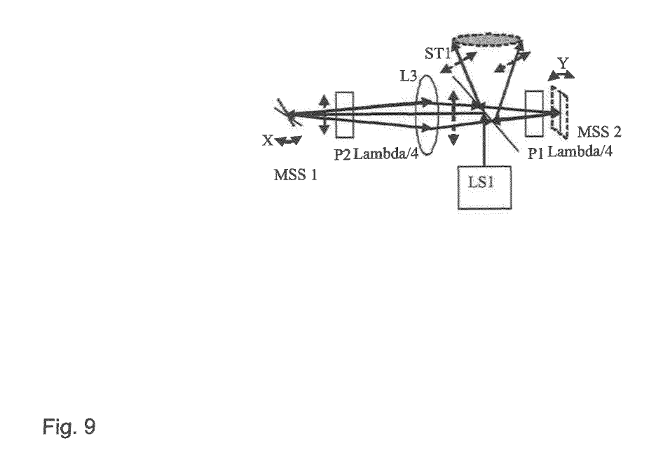

FIG. 9 a schematic block diagram of a second modification of the scanning micromirror of the preceding figures.

Parts that correspond to each other or are identical are denoted by the same reference symbols in the figures.

FIG. 1 provides a schematic block diagram of a first optical design of a projector device 1 as a first embodiment of the invention with an optional treatment BL.

The projector device 1 is designed to project a two-dimensional pattern 2 on a plane 3 which is defined by the retina in an eye 4. The two-dimensional pattern 2 projected on the retina of the eye 4 can be designed, for example, as an eye test character or accommodation target which supports the patient in adopting a defined position with his eye 4 and in accommodating the intraocular lens 5 to infinity during measurements or surgical treatment of his eye 4. This state of the eye 4 is considered as a reference state that makes very accurate measurements and exact treatment possible. Alternatively, the two-dimensional pattern may be used exclusively for measuring the eye 4.

The projector device 1 comprises a laser beam source LS1 which may be designed, for example, as a laser diode. The radiation emitted by the laser diode LS1 is selected such that the projector device 1 can be used for generating the eye test character in the visible range and with an intensity that ensures that the eye 4 is not injured. The beam diameter of the light beam emitted in the form of a laser beam by the laser beam source LS1 is preferably smaller than 500 μm, in particular smaller than 300 μm.

First, the laser beam traverses in a beam path 7 a polarization-dependent beam splitter ST2 and is linearly polarized by it. Subsequently the laser team is deflected at an angle of 90 degrees by an optionally polarization-dependent beam splitter ST1 which is arranged in the beam path. The laser beam then traverses first a lens L1 which may be designed as a glass or plastic lens and subsequently a lens EL with electrically controllable focal length which will also be referred to below as the adaptive lens EL. In its further path, the laser beam traverses a lambda-4 plate 6 and hits the center of a scanning micromirror MSS. The linearly polarized light beam is turned into circularly polarized light, which may be either right circularly polarized or left circularly polarized, by the lambda/4 plate. The scanning micromirror MSS facilitates a deflection of the laser beam at a deflection angle in two dimensions, so that the laser beam scans a surface. The mirror surface of the scanning micromirror is designed as a metallic mirror, so that the polarization of the laser beam changes after being reflected from e.g. right circularly polarized to left circularly polarized. On its way back, the laser beam again passes through the lambda-4 plate 6, the adaptive lens EL and the lens L1. Subsequently, the laser beam is split by the beam splitter ST1. In its further path, the laser beam passes through an eyepiece O1 and then enters the eye 4 and projects the two-dimensional pattern 2 on the plane 3.

However, the beam splitter ST1 uncouples a part of the returning laser beam from the beam path 7 of the laser beam and bounces it back in the direction of the laser beam source LS1. There the laser beam hits the polarization-dependent beam splitter ST2, which deflects it at an angle of 90° because the oscillation direction of the laser beam has been transposed by 90 degrees after passing through the lambda-4 plate 6 twice. Arranged after the polarization-dependent beam splitter ST2 is a lens L2, which may be designed either as a glass lens or as a plastic lens, said lens focusing the laser beam on a detector D1.

From a functional point of view, the projector device 1 can thus be subdivided into a region 8, which concerns the projection of the two-dimensional pattern 2 on the plane 3, and a region 9, which monitors the beam quality of the laser beam using the detector D1, as will be set forth below.

First, however, a description of the active components within region 8 will be presented:

The adaptive lens EL is an optical element with a controllable focal length. The control of the adaptive lens EL is performed via a driver T1. The electrically adaptive lens EL has a free diameter of up to 10 mm or of up to 2 mm, depending on the embodiment. In particular, the adaptive lens EL can only control the focal length but does not allow for multidimensional deformations in order to generate or correct higher-order optical aberrations. For this reason, the adaptive lens EL and the driver T1 can be constructed cost-effectively.

The scanning micromirror MSS is designed as an X-Y scanner and has a metallic mirror surface with a free diameter of, for example, 2 mm and facilitates a deflection of the incident laser beam at a deflection angle in two dimensions with frequencies ranging from 100 Hz to 110 kHz or more. In a particularly simple embodiment of the invention, the scanning micromirror MSS is controlled in a resonance mode so that it always performs the same motion sequence in a reproducible fashion. The motion sequence is chosen such that the laser beam incident at the center of the scanning micromirror MSS is guided by the deflection at the deflection angle such that it traces the surface area of the two-dimensional pattern 2 in a scanning or writing manner. The shape, outline or appearance of the two-dimensional pattern 2 is created by activating or deactivating (more generally: controlling) the laser beam source LS1, wherein said laser beam source is activated such that, for example, a ring or an image is projected as a two-dimensional pattern 2 on the plane 3.

In this example, the lens L1 is designed as a collimating lens which aligns the laser beam parallel to the optical axis of the beam path. Other embodiments of the lens L1 are possible as well. On its way from the laser beam source LS1 to the scanning micromirror MSS, the laser beam is transmitted through the center of the lens L1, while on the way from the scanning micromirror MSS to the plane 3, the laser beam may traverse the lens L1 outside the center or even in the peripheral region, depending on the deflection angle.

Together, the lens L2 and the detector D1 form a sensor device which makes it possible to determine the actual state of a wavefront of the laser beam. The detector D1 is designed as a flat detector such as a PSD (position sensitive diode), a CMOS camera, a CCD camera or a quadrant diode. The lens L2 is arranged at such a distance from a sensor surface of the detector D1 that all laser beams, irrespective of the deflection angle, ideally hit the same spot on the sensor surface, thereby forming a focal point. Preferably the lens L2 is a precision lens. If the beams shaped by the lenses EL and/or L1 already form such a focal point, the lens L2 can be dispensed with. With ideal optical components in the projector device 1, the focal length of the system would be independent of the location where the laser beam intersects the lenses EL and L1 and thus independent of the current deflection angle of the scanning micromirror MSS. However, with real optical components the focal length has local differences. In particular, the focal length tolerance also depends on the precision with which the optical components were manufactured, with possible production defects resulting in optical aberrations. Additional optical aberrations may result from a maladjustment of the optical components, in particular from tilting or something similar. If optical aberrations are present then the laser beam is no longer imaged in the common focal point on the sensor surface of the detector D1 but at a distance from said focal point. This results from the fact that the laser beam, owing to the optical aberrations, is no longer aligned in parallel to the beam axis after passing lens L1 and therefore is deflected differently by the lens L2. The distance between the actual point of incidence of the laser beam on the sensor surface and the ideal focal point on the sensor surface is thus a measure of the actual state of the wavefront of the laser beam, while the ideal focal point on the sensor surface represents the target state of the laser beam. Since the deflection angle of the laser beam caused by the scanning micromirror is known at all times, the detector D1 is able to register an actual state of the wavefront dependent on the deflection angle and thus in a position-resolved way.

Instead of the design of the sensor device described above, which comprises the detector D1 and the lens L2, it is also possible to use a wavefront measuring instrument to register the actual state of the wavefront of the laser beam or of the chronologically successive laser beams forming a laser beam bundle in a position-resolved way.

The difference between the actual state and the target state of the wavefront of the laser beam is evaluated by an evaluation unit 10 and then processed using a control device 11 or a regulator such that the wavefront of the laser beams is changed by controlling the adaptive lens EL via a driver T1 in such a way that the wavefront of the laser beams is transferred from the actual state to the target state. This correction of the wavefront, which will be referred to below also as tracking, correction or self-correction, is performed for each deflection angle of the scanning micromirror MSS, so that all laser beams directed at the eye 4 will have the target state of the wavefront, in particular a plane wavefront, after passing the lens L1.

In particular, the control of the adaptive lens EL and the control of the scanning micromirror MSS are synchronized in order to perform the self-correction in a position-resolved way or, in other words, dependent on the deflection angle. The measurement of the projector device 1 described above may optionally be performed only once when calibrating the device, in which case the timing-related and thus location-related connections between the control signals for the adaptive lens EL and the control signals for the scanning micromirror MSS are stored in the evaluation unit 10. Alternatively, the measurement can be carried out constantly during the operation of the projector device 1 and the self-correction, in the sense of a regulatory or control circuit, would be performed constantly as well.

In some embodiments, the laser beam source LS1 can be designed as being multicolored in order to project a multicolored two-dimensional pattern 2 on the plane 3. In this case, chromatic aberrations may be taken into account by the tracking or self-correction process as additional optical aberrations. The individual image pixels of the two-dimensional pattern 2 from the plane 3 are not displayed at the same time but sequentially, so the evaluation unit 10 regulates the control device 11 such that the compensation is performed by the adaptive lens EL as a function of the location of the laser beam or the deflection angle and as a function of the wavelength. In particular, the focal length of the whole system is kept constant as a function of the deflection angle and as a function of the wavelength.

Instead of using a control circuit, it is also possible to keep the focal length of the optical system constant via an analog or digital regulator which compares the actual value measured by the detector D1 as the actual state of the wavefront with the target value specified by the evaluation unit 10 as the target state of the wavefront. A possible embodiment is as follows: The control device 11 or the regulator amplifies, integrates and/or differentiates the difference between target value and actual value and uses the result to determine the control signal for the electrically controllable lens EL. If the center of the detector D1, which may be, for example, designed as a 2D position detector, is in the focal point of the whole optical system, the target value must be set to zero. The deviations from the zero position need to be compensated for by the control device 11 or the regulator, which means that the control signal must either be increased or decreased until the laser beam deflected at the detector D1 intersects at the zero position. In this way, all optical aberrations, including the chromatic aberrations if applicable, are corrected by the regulator 11.

The digital control may also be carried out by the evaluation unit 10. If the evaluation unit 10 is used for measuring the actual value, the evaluation unit 10 provides the control device 11 with both target values and actual values. The advantage of this design is that distortions can be minimized or eliminated using intelligent digital filtering of the actual value. This leads to a shorter transient process until the local focal length reaches the specified target value. In another possible embodiment, the optical system is replaced by an electronic hardware or software model (MEL). The local focal length of the optical system can be kept constant if an electronic model of the optical system in which the focal length is to be controlled is used as a software model for the optical system, said electronic model being designed in the form of an analog circuitry, consisting of one or several PT1 elements in the form of RC elements which are used as a low-pass filter, or a programmed differential or difference equation. The model describes the temporal behavior of the focal length of the whole optical system, consisting of the adaptive lens EL, the driver T1 of the lenses and the lens L1.

To provide the evaluation unit 10 continuously with an actual state of the wavefront of the laser beam, the laser beam source LS1 used may be optionally supplemented or replaced by an IR light source which emits an invisible laser beam in the infrared range and which is switched on permanently or alternating with the laser beam source LS1. The IR light source allows for registering an actual value of the wavefront even when a dark, i.e. black, image content needs to be displayed as the two-dimensional pattern 2 and the laser light source LS1 is accordingly switched off in the visible range.

Starting from the plane 3, the laser beam is scattered back or reflected, hits the beam splitter ST1 again and is guided into a further sensor device W1 which is also designed to determine the actual state of the wavefront of the laser beam. The further sensor device W1 is thus arranged at the end of the beam path, in particular behind the plane 3 if viewed in the direction of the beam, and therefore records all optical aberrations accumulated in the beam path by analyzing the laser beam. In particular, the sensor device W1 records optical aberrations of the eye 4 as well. The sensor device W1 can be designed as a Shack-Hartmann sensor, a Tschernig aberrometer or a wavefront measuring instrument scanning the eye 4.

In a first simple embodiment of the invention, the measurement results of the sensor device W1 are interpreted as the local refractive power of the eye 4 and passed on. This embodiment is advantageous because the self-correction of the projector device 1 guarantees that the laser beam is guided into the eye 4 e.g. in parallel to the beam axis of the beam path and that all occurrent optical aberrations have been introduced by the eye 4 as an optical element.

In a second embodiment of the invention, the sensor device W1 feeds the actual state of the wavefront of the laser beam into the evaluation unit 10 where it serves as input value for a regulatory or control circuit.

In this second embodiment, the tracking or self-correction is performed based on the actual state of the laser beam as recorded by the sensor device W1. If the evaluation unit 10 is designed such that deviations between the actual state and the target state are compensated for based on the measurement readings of the sensor device W1, then it is guaranteed, on the one hand, that the two-dimensional pattern 2 on the plane 3 is indeed in sharp focus. In the case that the two-dimensional pattern 2 is designed as an eye test character or accommodation target, this embodiment is advantageous because the patient will recognize a clear eye test character. In the case that a laser beam for surgical treatment of the eye, e.g. the retina, is emitted by the laser BL and guided along the same axis as the laser beam from the laser beam source LS1, this embodiment is advantageous because the laser beam from the laser BL will also be displayed in sharp focus and in the correct position on the plane 3 and thus on the retina. When the treatment laser beam from the laser BL and the laser beam superimpose, both beams are guided through the self-corrected or tracked beam path and displayed on the plane 3, in particular on the retina, in sharp focus. The laser BL may be used, for example, to weld the retina or, if the plane 3 is transposed into the intraocular lens, to treat the intraocular lens. On the other hand, a joint evaluation of the actual states of both the sensor device D1/L2 and the further sensor device W1 allows for the assignment of the measured optical aberrations to either the projector device 1 or the eye 4, so that those optical aberrations that are assigned to the eye 4 also constitute an exact measurement of the local refractive power of the eye 4.

The projector device 1 can thus be used in the following modes of operation:
1. Self-correction of the optical system for the partial region of the beam path up to the beam splitter ST1 by means of the evaluation unit 10, based on the measurement readings from the detector D1 as actual state of the wavefront of the laser beam or the laser beams. Determination of the local refractive power of the eye 4 by means of the sensor device W1.
2. Self-correction of the optical system for the beam path from the laser beam source LS1 up to behind the plane 3 by means of the evaluation unit 10, based on the measurement readings from the sensor device W1. Determination of the local refractive power of the eye 4 by comparing the measurement readings from the detector D1 with those from the sensor device W1.
3. Self-correction of the optical system for the beam path from the laser beam source LS1 up to behind the plane 3 by means of the evaluation unit 10, based on the measurement readings from the sensor device W1. In the case that the detector D1 is left out in a further embodiment, the advantage of that embodiment would be the display of the eye test character in sharp focus.

It is optionally possible to utilize additional beam splitters ST3 and ST4 to couple a further laser beam source LS2 into the beam path.

FIG. 2 shows a second embodiment of the invention. The main difference from the embodiment shown in FIG. 1 is that the beam path from the laser beam source LS1 to the scanning micromirror MSS is different. In this embodiment, the laser beam is guided via a polarization-dependent beam splitter ST3 onto the scanning micromirror MSS such that the laser beam is deflected by the scanning micromirror MSS at an angle of 90°. This is advantageous because the incident beams need not be guided through the whole optical system, which prevents the optical system from shaping the beams and/or generating loss through reflection and scattering. The various methods of regulating and/or controlling the correction of optical aberrations work exactly like in the embodiment described according to FIG. 1.

In this embodiment, the image size of the two-dimensional pattern 2 on the plane 3 may be additionally influenced by changing the distance between the scanning micromirror MSS and the adaptive lens EL or the distance between the adaptive lens EL and the lens L1. This also causes the treatment and/or projection beams hitting the eye 4 to scan the eye 4 at a different angle dependent on the distance set. For example, the scanning micromirror MSS in conjunction with the beam splinter ST3 and the laser beam source LS1 and/or the adaptive lens EL may be mounted on a motorized slide which is movable along the optical axis of the lens L1 as indicated by arrow A.

The illustration in FIG. 3 shows a further embodiment of the invention, wherein the laser beam of the laser beam source ST1 and the treatment beam of the laser BL are combined via mirrors ST3 and ST4 and deflected via a deflecting mirror US1 to the scanning micromirror MSS. The scanning micromirror MSS and the deflecting mirror US1 are arranged on a slide M1 that can be moved in the direction indicated by arrow A in order to facilitate an adjustment of the distance between the scanning micromirror MSS and the lens L1, so that by moving the slide M1 the mean ametropia of the eye, the sphere, can be compensated for, so that e.g. the image size in the eye 4 reaches a defined size of approximately 1×1 mm².

For the embodiments already illustrated and those described subsequently, the following optional method for setting the measuring distance is proposed: Since the measurement accuracy and the local assignment of the measurement results of a diagnostic device in ophthalmology depends on the distance between the eye 4 and the sensor device W1, the optical component O1 generates a small focal point on the apex of the cornea of the eye 4 that is located exactly at the desired measuring distance. If the eye 4 is not located in the focal point thus generated, a spot of small or large dimensions will appear on the cornea. This spot is evaluated using the observation camera integrated in the sensor device W1 so that tire exact measuring distance is shown to the user. The exact measuring distance is reached when the focal point has reached its minimum. The optical component O1 can be designed as an optical component with electrically controllable focal length such as a fluid lens objective or a lens controlled by electroactive polymers (EAPs). For this purpose, a suitable driver T2 is required which receives its control signals from the evaluation unit 10. The focal point for the measuring distance is used only for the alignment of the projector device 1. While an ametropia is measured or treated, the optical component O1 is inactive, which means that the evaluation unit 10 sets the longest focal length, if possible 0 dioptres which equals a focal length of ∞ mm. The optical component O1 may be alternatively designed as a standard lens with a free aperture or with a free opening of 10 mm or more, so that the optical component O1 only focuses the peripheral projection beams generated by the scanning micromirror MSS at a focal point at the desired measuring distance. Suitable for this purpose are reflecting, diffracting and/or refracting optics in the form of a sleeve.

An advantage of the embodiments illustrated in FIGS. 1 to 3 and in the Figures described below is that the focal point for determining the measuring distance is generated by the same light sources LS1 or LS2 which project the two-dimensional patterns.

FIG. 4 illustrates a further form of the invention wherein, in addition to the laser beam source LS1, a further laser beam source LS2 is coupled into the beam path along the same axis as the laser beam source LS1. The laser beams of the two laser beam sources LS1 and LS2 differ in their polarization. The different polarization makes it possible to split the beam path into two different beam paths using a polarizing mirror PST1, so that the first laser beam source LS1 only projects into one eye 4 and the laser beam source LS2 only projects into the other eye 4. This is advantageous because it allows the projector device 1 to provide both eyes 4 simultaneously or in parallel with two-dimensional patterns 2 which may be different, if so desired.

In the constructional embodiment, the laser beams of the laser beam sources LS1 and LS2 and, if applicable, of the treatment laser BL are guided along the same axis, as has been already explained above in connection with FIG. 1. Behind the beam splitter ST1, the laser beams are again split by the polarization-dependent beam splitter PST1, according to their polarization, into two separate beam paths. Each beam path is then guided via a deflecting mirror US2 or US3 and an eyepiece O2 or O3 to the respective eye 4. The back reflections of the laser beams and/or the scattered light from the eyes 4 are again projected onto the sensor device W1, so that an actual state of the wavefront of the laser beam or the laser beams emitted by the corresponding laser beam source LS1 and LS2 can be recorded for each eye 4 by said sensor device.

The embodiment shown in FIG. 4 is thus an extension of the projector devices 1 illustrated in the preceding FIG. 2. This extension facilitates stereoscopic vision, in other words, 3D vision, which can be utilized, for example, to measure ametropia in both eyes under natural conditions. To achieve this, the two-dimensional patterns 2 generated by the scanning micromirror MSS need to be optically separated so that the two-dimensional pattern 2 intended for one eye 4 can only be seen by that eye 4.

If the two two-dimensional patterns 2 are created by linearly polarized projection beams with different polarization directions which are emitted by the laser beam sources LS1 and LS2, then it is possible to separate the two two-dimensional patterns 2 using the polarization-dependent beam splitter or polarized mirror PST1. Beams with perpendicular polarization are deflected onto the right eye 4 and beams with parallel polarization are deflected onto the left eye 4 or vice versa. The two two-dimensional patterns 2 are generated simultaneously by modulating the respective laser beam sources LS1 and LS2. The projector device generates both two-dimensional patterns 2 with the same and maximum possible resolution and the maximum refresh rate, for example, so as to make sure that the image generation of the two-dimensional patterns 2 by scanning the eye 4 is not noticed by the patient. It is also possible to separate the two two-dimensional patterns 2 by including polarization-dependent filters in the eyepieces O2 and O3, so that, for example, the right eyepiece O3 can only be passed by beams with perpendicular polarization and the left eyepiece O2 is only transparent to light with parallel polarization.

In another embodiment, the two-dimensional patterns 2 for the right and the left eye 4 may also be projected by a common scanning micromirror MSS in a sequential manner, i.e. in rapid succession, into the eye 4, using just one of the light sources LS1 or LS2. To achieve the 3D effect, shutters would be integrated into both eyepieces O2 and O3. These shutters allow light to pass in turns and only if the respective two-dimensional pattern 2 for the right or the left eye 4 is to be created. The shutters are transparently operated by the evaluation unit 10 synchronously to the image generation for the respective eye 4. The advantage of this embodiment is that only a single light source is needed to generate both two-dimensional patterns 2, said light source being any monochromatic or multicolored light source, in particular an RGB light source. However, while the resolution of the two-dimensional patterns 2 would be sufficient, the refresh rate would be only half as high as in the example with polarization-dependent laser beam sources LS1 and LS2.

The eyepieces O2 and O3 each have at least one lens with electrically controllable focal length. In particular, the eyepieces O2 and O3 have an adaptive lens which is constructionally identical to any constructional variant of the adaptive lens EL. The evaluation unit 10 controls or regulates the focal length of the eyepieces O2 and O3 such that both eyes 4 create a sharp image on the retina. Local optical aberrations of the respective eye 4 are corrected by the eyepieces O2 and/or O3 with electrically controllable focal length individually for the respective eye 4, applying the same methods and devices described above in connection with FIG. 1. Error-free imaging makes it possible to perceive a sharp 3D image.

The embodiment shown in FIG. 4 therefore implements three supervisory circuits, in particular regulatory or control circuits:
1. Self-correction of the beam path up to the beam splitter ST1 by determining the actual state of the wavefront of the laser beam emitted by the laser beam source LS1 or LS2 by means of the detector D1 and by controlling the adaptive lens EL.
2. Self-correction of the beam path including the eye 4 by determining the actual state of the wavefront of the laser beam from the laser beam source LS1 using the sensor device W1 and by controlling the adaptive lens in the eyepiece O3.
3. Self-correction of the beam path including the eye 4 by determining the actual state of the wavefront of the laser beam from the laser beam source LS2 using the sensor device W1 and by controlling the adaptive lens in the eyepiece O2.

Depending on the design of the polarized mirror PST1 and of the laser beam sources LS1 and LS2, the eyepieces O2 and O3 may be interchanged in the control circuits.

The distance between the eyes of the individual patient must be taken into account when performing binocular measurements. For this reason it is possible to shift the lateral distance between the eyepiece O2, which is rigidly connected to the deflecting mirror US2, and the eyepiece O3, which is rigidly corrected to the deflecting mirror US3, in the y direction. In order to achieve a high measurement accuracy, the measuring distance between the eye and the wavefront sensor W1 needs to be kept constant. This means that if the distance between the eyepiece O2 and the eyepiece O3 is decreased, it is necessary to increase the distance between the eyepiece O2 and the eye in the z direction by the same amount, and vice versa. The measuring distance can be checked using the optical component O1, as has been described above. The eyepieces O2 and O3 are always moved by the same distance to the optical axis to ensure that the whole construction remains symmetrical to the optical axis.

FIG. 5 shows a modification of the embodiment shown in FIG. 4 wherein the beam path in front of the scanning micromirror MSS is designed analogously to FIG. 1.

FIG. 6 shows a possible further form of the sensor device W1 of the preceding figures wherein the detector D1 is left out. In FIG. 6, an exemplary application of the projector device 1 in a wavefront measuring instrument is illustrated. The eyepieces O2 and O3 compensate for the focal optical aberrations of the eye, as described with respect to FIGS. 4 and 5. The scanning micromirror MSS, which is mounted on a motorized slide which is movable in the 2 direction, is positioned at a specific distance from the eyepieces O2 and O3. The distance from the eyepiece and the adjustable deflection angle of the scanning micromirror MSS ensure that the surface of the eye 4 can be completely scanned on an area of 10×10 mm². With the set focal length of the two eyepieces O2 and O3, the laser beams emitted by the laser beam sources LS1 and/or LS2 are refracted at a defined angle, so that an error-free two-dimensional pattern 2 of size 1×1 mm², in particular an image, is displayed sharply on the plane 3 of the retina. The laser beam scattered by the retina of the eye 4 which leaves the eye 4 in close proximity to the apex of the eye 4 is registered by a detector D2 . The detector D2 measures the angle of said laser beams and calculates the local ametropia based on the measurement. To ensure the reproducibility of the measurement results and a consistently high measurement accuracy over the entire measurement range, a diaphragm B1 can be displayed exactly on the cornea of the eye 4, preferably exactly within the visual axis of the eye, through the eyepiece O2 or O3 and through the optical component O4 located before the diaphragm B1. The displayed diaphragm B1 guarantees that only those laser beams are evaluated which leave the eye 4 in the vicinity of the apex of the eye 4 through an aperture with a diameter of e.g. 1 mm. Thus only those laser beams are evaluated which are only marginally refracted by the layers of the eye on their way from the plane 3 of the retina to the cornea (paraxial beams). In this example, the eyepieces O2 and O3 are designed as optical components with electrically controllable focal length which are used to correct the optical aberrations of the optical system of the eye 4. The focal length of the eyepieces O2 and O3 is controlled such that the laser beam intersecting the cornea at a particular location enters the eye at an incidence angle that ensures an error-free display on the retina. The temporal change of the focal length of the eyepieces O2 and/or O3 compensates for the local ametropia of the eye 4. This change of the focal length of the eyepieces O2 and/or O3, however, causes a temporal change in the location where the diaphragm B1 is displayed, thus distorting the measurement result. This disadvantage is compensated for using the optical component O4 which, like the eyepieces O2 and O3, is designed as an optical component with electrically controllable focal length, by controlling the focal length of the optical component O4 synchronously to the focal length of the eyepiece O2 or O3, depending on which eye 4 is to be measured. In other words, if the focal length f of the eyepiece O2 is changed by ±Δ f, then the focal length of the optical component O4 needs to be changed by the same value ±Δ f at the same time if, for example, the optical design of the eyepieces O2 and O4 is identical.

FIG. 7 illustrates a very compact embodiment of a projector device 1. The embodiment can be reduced to the laser beam sources LS1 and, if required, LS2, the scanning micromirror MSS, the adaptive lens EL with electrically controllable focal length and the evaluation unit 10. The lens L2 and the detector D1 are used just once during the calibration of the projector device 1. The evaluation unit 10 stores the control signal for the focal length of the lens EL, which was measured during calibration. Using this value, the evaluation unit 10 controls the focal length of the lens EL, while the scanning micromirror MSS deflects the laser beams so that images are projected without optical aberrations.

FIG. 8 describes a replacement device for the scanning micromirror MSS of the preceding figures. The objective is to generate the two-dimensional patterns 2 using individual beams which start from a point source and form a defined angle with the optical axis, so that, for example, a rectangular area can be scanned completely. For this purpose, two one-dimensional scanners MSS1 and MSS2 are utilized. The scanner MSS1 oscillates in the x direction and the scanner MSS2 oscillates in the y direction. The laser beam emitted by the laser beam source LS1 first hits, for example, the scanner MSS1 which oscillates in the x direction. Said scanner deflects the projection beam at an angle α in the x direction. To achieve the advantages of a point source, or to replicate the advantages of a 2D scanning micromirror, the laser beam reflected by the scanner MSS1 is focused onto the one-dimensional scanner MSS2 by the lens L3 which, for example, may be designed as an asphere. The scanner MSS2 oscillates in the y direction and additionally deflects the projection beam at an angle β in the Y direction. The projection beam thus receives a deflection in the x and y directions and fully replaces a 2D scanning micromirror. If the scanners MSS1 and MSS2 are not designed as resonance oscillators but as galvano scanners then each point of the projection surface can be controlled at any given time and for as long as the application requires.

To prevent multiple reflections between the scanning mirrors MSS1 and MSS2, the light source LS1 is designed as a linearly polarized light source. The polarization-dependent beam splitter ST1 lets one polarization direction pass and reflects the other polarization direction. To ensure that the beam bundle is not reflected back onto the scanner MSS1 by the scanner MSS2, the polarization direction of the projection beam is rotated by 90 degrees by the λ/4 plate P Lambda/4. On the way to the scanner MSS2, the λ/4 plate P Lambda/4 generates circularly polarized light, for example right circularly polarized light, which is reflected by the scanner MSS2, which is designed as a metallic mirror. On the way from the scanner MSS2 to the beam splitter ST1, the light, which is now e.g. left circularly polarized because of the reflection at the metallic mirror, is transformed by the λ/4 plate P Lambda/4 into linearly polarized light. The polarization direction thus rotates from, for example, perpendicular to parallel or vice versa.

FIG. 9 illustrates a further replacement device for the 2D scanning micromirror. The laser beam LS1 with e.g. perpendicular polarization is reflected by the polarization-dependent beam splitter ST1 onto the scanning micromirror MSS1. After two passes, the λ/4 plate P1 lambda/4 has rotated the polarization direction from e.g. perpendicular to parallel, so that the beam splitter ST1 lets the projection beam, which has been focused by the lens L3, pass to the 1D scanning micromirror MSS2.

The focal point lies on the scanning micromirror MSS2. The λ/4 plate P2 Lambda/4 rotates the polarization direction from e.g. parallel to perpendicular, so that the projection beam reflected by the scanning micromirror MSS2 is reflected in two dimensions in an upward direction by the beam splitter ST1.

The λ/4 plate P1 Lambda/4 or P2 Lambda/4 generates circularly polarized light from a linearly polarized projection beam and linearly polarized light from circularly polarized light. In doing this it rotates the polarization direction of the linearly polarized projection beam from e.g. perpendicular polarization to parallel polarization or vice versa, since the non-depolarizing scanning micromirror MSS1 or MSS2 generates e.g. left circularly polarized light from right circularly polarized light or vice versa by reflecting it.

### REFERENCE SYMBOL LIST

1 projector device
2 pattern
3 plane
4 eye
5 intraocular lens
6 lambda-4 plate
7 beam path
8 region
9 region
10 evaluation unit (digital processor/control)
11 regulator
B1 diaphragm
W1 wavefront measuring instrument/ametropia measuring instrument
LS1 projection beam source (RGB laser diode or SLD and IR), parallel polarization
LS2 projection beam source (RGB laser diode or SLD and IR), perpendicular polarization
BL treatment laser
L2 lens (glass or plastic lens)
L2 lens (glass or plastic lens)
L3 lens (glass or plastic lens)
EL lens with electrically controllable focal length
MEL electronic model of the electrically controllable lens
MSS 2D micro-scanning mirror
MSS1 1D micro-scanning mirror
MSS2 1D micro-scanning mirror
PST1 polarization-dependent beam splitter
ST1 beam splitter
ST2 beam splitter
ST3 beam splitter
ST4 beam splitter
US1 deflecting mirror
US2 deflecting mirror
US3 deflecting mirror
P Lambda/b 4 λ/4 plate
O1 eyepiece with electrically controllable focal length
O2 eyepiece with electrically controllable focal length and/or shutter
O3 eyepiece with electrically controllable focal length and/or shutter
O4 objective with electrically controllable focal length
D1 detector (PSD or CCD/CMOS camera or wavefront measuring instrument)
D2 detector (PSD or CCD/CMOS camera)
T1 driver for the electrically controllable lenses
T2 driver for the electrically controllable eyepieces
T3 driver for the electrically controllable eyepieces
T4 driver for the electrically controllable eyepieces
T5 driver for the electrically controllable eyepieces

1. A projector device for projecting a two-dimensional pattern on a plane, in particular in a human eye,
with at least one light source (LS1, LS2) which generates a light beam,
with a deflecting device (MSS) which facilitates a deflection of the light beam at a deflection angle in order to generate the two-dimensional pattern on the plane,
with a beam path which extends at least from the light source (LS1, LS2) to the plane,
with at least one sensor device (L1, D1, W2, D2) which is designed to determine the actual state of a wavefront of the light beam or its backscattering at any position of the beam path,
with at least one correcting device (EL; O2, O3) which is arranged in the beam path and which facilitates an alteration of the wavefront of the light beam,
with an evaluation unit which is designed to record the actual state of the wavefront and to compare the actual state of the wavefront of the light beam with a target state of the wavefront of the light beam,
wherein:
the correcting device comprises an optical control element (EL, O2, O3) with controllable focal length, wherein the evaluation unit is designed, due to its programming and/or wiring, to control the optical control element (EL, O2, O3) such that the actual state tracks the target state in a position-resolved way, in particular location-dependent with respect to the plane and/or angle-dependent with respect to the deflection angle.

2. The projector device of claim 1, wherein the two-dimensional pattern is designed as an accommodation target for the eye.

3. The projector device of claim 1, wherein the deflecting device is designed as a scanning micromirror (MSS).

4. The projector device of claim 1, wherein the real-time tracking is achieved exclusively by controlling the optical control element (EL, O2, O3).

5. The projector device of claim 1, wherein the light source (LS1, LS2) is designed as a laser beam source.

6. The projector device of claim 1, wherein the light source is designed to emit colored and/or polarized light.

7. The projector device of claim 1, wherein the at least one sensor device (L2, D1; W1; D2) is arranged in the beam path such that it receives a backscattering of the light beam from the plane.

8. The projector device of claim 1, wherein the at least one sensor device (L2, D1) or a further sensor device (L2, D1) is arranged in the beam path before the plane such that it receives a light beam incident towards the plane.

9. The projector device of claim 8, wherein the at least one sensor device (L2, D1) or the further sensor device (L2, D1) is arranged in the beam path after a collimating lens L1.

10. The projector device of claim 1, wherein the at least one sensor device (L2, D1; W1; D2) is designed as a wavefront measuring instrument.

11. The projector device of claim 1, wherein the at least one sensor device (L2, D1; W1; D2) comprises a position-resolving sensor, in particular a PSD, a CCD camera or a CMOS camera.

12. The projector device of claim 1, wherein the projector device is designed as a binocular device and facilitates a parallel projection of two-dimensional patterns into both eyes of a patient.

13. The projector device of claim 1, wherein the at least one correcting device (EL, O2, O3) is arranged before a collimating lens (L1) and/or in an eyepiece (O2, O3) of the projector device.

14. The projector device of claim 1, wherein the light beam is an invisible laser beam or comprises said invisible laser beam.

15. A medical device with a projector device of claim 1, designed as a topography measuring instrument, a wavefront measuring instrument, a layer thickness measuring instrument or a treatment laser system.

16. The medical device of claim 15, wherein the treatment laser (BL) of the treatment laser system is guided along the same axis as the light beam.
